(19) United States
(12) Patent Application Publication
(10) Pub. No.: US 2009/0012170 A1
(43) Pub. Date: Jan. 8, 2009

(54) TREATMENT OF SYMPTOMS OF MOTOR DYSFUNCTION

(76) Inventors: Helena Nissinen, Espoo (FI); Mikko Vahteristo, Kuopio (FI); Mikko Kuoppamaki, Raisio (FI); Juha Ellmen, Turku (FI); Mika Leinonen, Stockholm (SE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/067,070

(22) PCT Filed: Sep. 20, 2006
(86) PCT No.: PCT/FI06/00306

§ 371 Date: Jul. 10, 2008

Related U.S. Application Data
(60) Provisional application No. 60/718,741, filed on Sep. 21, 2005.

(51) Int. Cl. A61K 31/197; A61K 31/277; A61P 25/00

(52) U.S. Cl. 514/567; 514/619

(57) ABSTRACT

The present invention relates to the use of a COMT inhibitor in combination with a dopamine precursor and a peripheral decarboxylase inhibitor in the treatment of motor dysfunctions.

The present invention relates to the use of COMT inhibitors in combination with dopaminergic agents in the treatment of the symptoms of motor dysfunction. In one embodiment, the invention relates to the use of entacapone in combination with levodopa and a peripheral decarboxylase inhibitor in maintaining the motor functioning of patients suffering from Parkinson's disease.

### BACKGROUND OF THE INVENTION

Parkinson's disease (PD) is a chronic disease that causes patients motor dysfunction such as tremor, bradykinesia, rigidity and difficulties in speech and in the initiation of motor actions. It is caused by the deprivation of dopamine in the brain. In addition to Parkinson's disease, there are also other conditions connected with motor dysfunction which may be ascribed to dopaminergic malfunction, e.g. restless legs syndrome (RLS), which is characterized by an irresistible urge to move the legs (akathisia).

The insufficiency of dopamine in the brain may be compensated by administering to the patient dopaminergic agents, such as dopamine agonists or dopamine precursors, e.g. levodopa. There is no established cure for Parkinson's disease, which means that the symptoms worsen, necessitating an increase in daily dosage of the medicament as the disease progresses. Furthermore, the chronic use of increased dosages of levodopa leads to the development of motor complications, such as wearing off and involuntary movements (dyskinesia).

The symptoms of motor dysfunction can be improved by levodopa treatment especially combined with compounds that improve its efficacy. This has been done by combining with levodopa other medicaments that prevent the metabolism of levodopa in the periphery, such as peripheral decarbocylase (DDC) inhibitors and catechol-O-methyl transferase (COMT) inhibitors.

There are several publications reporting the use of entacapone in combination with levodopa (and a DDC inhibitor) in the context of increasing “ON-time”, reducing “OFF-time” and improving Parkinson's disease symptoms measured by the Unified Parkinson's Disease Rating Scale (UPDRS). The rationale for this phenomenon is believed to be the fact that the effect of levodopa may be prolonged and enhanced when entacapone (and a DDC inhibitor) is used and at the same time the levodopa dose can be reduced. According to the knowledge of the present inventors there are, however, no earlier reports suggesting that the early use of a COMT inhibitor in combination with levodopa and a DDC inhibitor could maintain the motor functioning of the patients suffering from Parkinson's disease better than delayed use of the specified combination. The finding of the present inventors that patients with Parkinson's disease treated with levodopa would gain heightened or optimal benefit from such an early use is therefore considered to be new and surprising.

### SUMMARY OF THE INVENTION

Applicants have discovered that the progression of the symptoms of motor dysfunctions in PD patients receiving levodopa therapy may be delayed significantly by an early introduction of a COMT inhibitor in said therapy instead of a delayed introduction.

It is one aspect of the invention to provide a method for the treatment of the symptoms of motor dysfunctions in a mammal, comprising administering to the mammal in need of the treatment an effective amount of a catechol-O-methyltransferase inhibitor, a dopamine precursor and a peripheral decarboxylase inhibitor.

It is a further aspect of the invention to provide a method of delaying the progression of the symptoms of motor dysfunctions in a mammal, comprising administering to the mammal in need thereof an effective amount of a catechol-O-methyltransferase inhibitor, a dopamine precursor and a peripheral decarboxylase inhibitor.

It is a further aspect of the invention to provide a method of delaying the progression of the symptoms of motor dysfunctions in a mammal by administering entacapone, levodopa and a peripheral decarboxylase inhibitor.

It is a further aspect of the invention to provide a method of delaying the progression of the symptoms of motor dysfunctions in a mammal by administering entacapone or its pharmaceutically acceptable salts and esters, levodopa and carbidopa.

It is a further aspect of the invention to provide a method of the treatment of delaying the progression of the symptoms of motor dysfunctions in a mammal by administering entacapone, levodopa and benserazide.

### BRIEF DESCRIPTION OF THE DRAWINGS

FIG. 1 shows the motor function of patients with Parkinson's disease measured by the Unified Parkinson's Disease Rating Scale (UPDRS) part III (Motor examination) from the start of entacapone treatment combined with levodopa/DDCI or with levodopa/DDCI combined with placebo in a double blind study. From 6 months onwards the patients continued in an open follow-up study and all received entacapone combined with levodopa/DDCI treatment i.e. those switched from placebo (top line) and those continuing with entacapone.

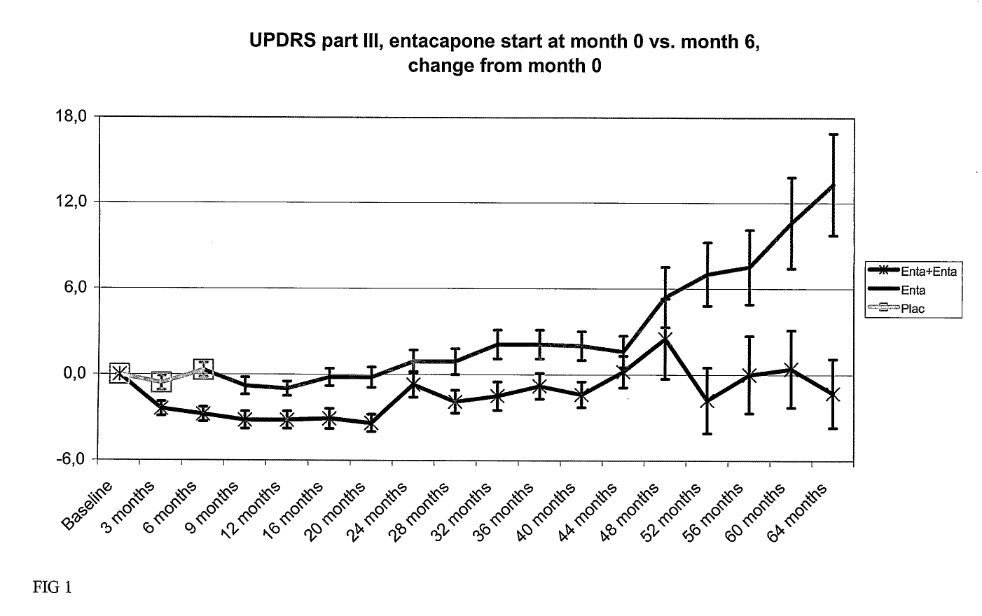

FIG. 2 shows daily levodopa dose of patients with Parkinson's disease from the start of entacapone treatment combined with levodopa/DDCI or with levodopa/DDCI combined with placebo in a double blind study. From 6 months onwards the patients continued in an open follow-up study and all received entacapone combined with levodopa/DDCI treatment i.e. those switched from placebo and those continuing with entacapone (line marked “Enta+Enta”).

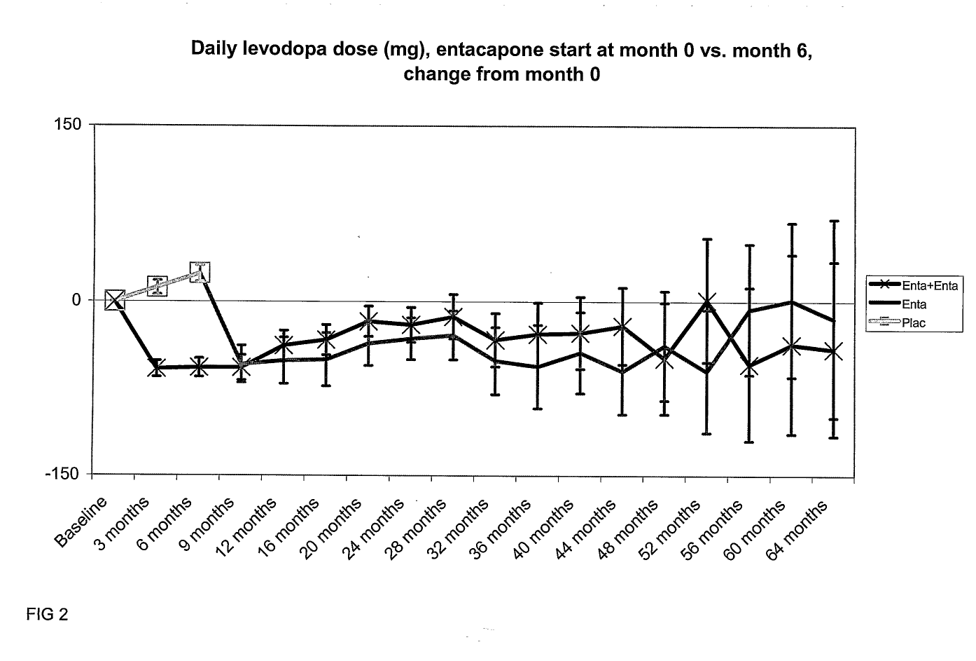

### DETAILED DESCRIPTION OF THE INVENTION

Applicants have now found that it is possible to delay the progression of the symptoms of motor dysfunctions in Parkinson's disease patients such as tremor, bradykinesia or rigidity by an early administration to the patients of a COMT inhibitor in combination with a dopamine precursor and a peripheral DDC inhibitor.

To achieve optimal results, the treatment with entacapone is started at the same time as the treatment with the dopamine precursor. In order to be effective, for example in the treatment of Parkinson's disease or Restless Legs Syndrome, each dosage form according to the invention is to be taken sequentially or simultaneously with a dopamine precursor (for example levodopa or its prodrug such as ethyl ester of levodopa) and a DDC inhibitor and optional other drug substances.

The pharmacologically effective amounts of entacapone, levodopa and the DDC inhibitor are dependent on numerous factors known to those skilled in the art, such as the type and the severity of the condition of the patient, the highest recommended daily dose being 200 mg of entacapone ten times a day (i.e. 2000 mg entacapone per day). For example, in the treatment of RLS the daily dose of levodopa can be as low as 50 mg, for example from 50 mg to 300 mg but can be from 200 mg to 600 mg, divided into 1 to 4, preferable into 1 to 2 individual doses, whereas in the treatment of severely ill Parkinsonian patients the daily dose of levodopa can be considerably higher, for example from 100 mg to 2000 mg divided, for example from two to ten individual doses.

Entacapone ((E)-2-cyano-3-(3,4-dihydroxy-5-nitrophenyl)-N-diethyl-2 propenamide) is a catechol-O-methyl transferase (COMT) commercially available as a stand-alone formulation under the trademarks Comtess® and Comtan®.

The amount of entacapone in a single dosage unit according to the invention is, for instance from 100 mg to 400 mg, e.g. from 100 mg to 300 mg, especially from 100 mg to 200 mg. The amount of levodopa is, for instance from 50 mg to 400 mg, e.g. from 50 mg to 300 mg, for example from 50 mg to 200 mg.

DDC inhibitors include, without limitation, carbidopa and benserazide. Levodopa and carbidopa are commercially available both as immediate release and slow release (depot) combination tablets sold in Europe under, for instance, the following trademarks: Nacom®, Sinemet®, Sinemet Depot® and Sinemet® Plus. Levodopa and benserazide are commercially available both as immediate release and slow release (depot) combination tablets, for instance, under the trademark Madopar® and Rextex®.

The amount of carbidopa is, for instance from 5 mg to 200 mg, e.g. from 5 mg to 100 mg, e.g. from 5 mg to 50 mg.

The DDC inhibitor and levodopa (or other dopamine precursor) are administered, for example in a ratio of from 1:1 to 1:40, for example from 1:4 to 1:10.

It is also possible to administer the COMT inhibitor, dopamine precursor and DDC inhibitor in a fixed combination. A fixed combination of entacapone, levodopa and carbidopa is available on the market under the trademark Stalevo® (levodopa:carbidopa entacapone: 50 mg:12.5 mg:200 mg, 100 mg:25 mg:200 mg and 150 mg:37.5 mg:200 mg).

The invention will be further clarified by the following example, which is intended to be purely exemplary of the invention.

### EXAMPLE

A study was carried out to evaluate the effect of earlier vs. delayed start of entacapone adjunct to traditional levodopa/DDC inhibitor treatment on the long-term Parkinson's disease symptom control up to 5 years.

Methods

Analysis of pooled data of three pivotal double blind, placebo-controlled studies, and their respective long-term open-label extension studies up to 5 years on entacapone adjunct to optimised levodopa/DDC inhibitor and other PD treatment.

A total of 488 PD patients, of whom 269 were on entacapone and 219 on placebo during the 6-month double blind phase, continued with open entacapone treatment in the long-term extension phases up to 5 years. UPDRS III assessment and the mean daily levodopa dose were reported until the point when a minimum of 10 patients in each treatment arm was available, i.e. up to five years.

Results

At the baseline of the double-blind phase, the mean (SD) age of patients included in this analysis was 61.8 (9.2) years, the duration of PD 9.9 (5.1) years, the duration of levodopa treatment 8.2 (4.6) years and the mean daily levodopa dose being 682 (344) mg.

The benefit of earlier start of entacapone adjunct to traditional levodopa/DDC inhibitor measured by UPDRS III was maintained during the whole analysed treatment period up to 5 years. The mean difference in UPDRS III over time between the earlier and delayed start treatment groups was −1.66, 95%CI [−3.01, −0.31]; Repeated measures analysis; p<0.05.

In the delayed start group, the mean daily levodopa dose decreased after initiation of open entacapone by about 70 mg, which is comparable with the decrease in the earlier start group at the beginning of the double-blind phase, staying thereafter in both groups consistently below the baseline.

In conclusion, the better symptom control measured by UPDRS III was maintained up to 5 years in the PD patient group starting entacapone adjunct to traditional levodopa/DDC inhibitor 6 months earlier compared with the delayed start group.

These results clearly support the early introduction of entacapone in the levodopa therapy in contrast to the current treatment recommendations.

1. A method for the treatment of the symptoms of motor dysfunctions in a mammal, comprising administering to the mammal in need of the treatment an effective amount of a catechol-O-methyltransferase inhibitor, a dopamine precursor and a peripheral decarboxylase inhibitor.

2. The method according to claim 1 comprising delaying the progression of the symptoms of motor dysfunctions.

3. The method according to claim 1, wherein the COMT inhibitor is entacapone or its pharmaceutically acceptable salt or ester.

4. The method according to claim 1, wherein the dopamine precursor is L-dopa.

5. The method according to claim 1. wherein the peripheral decarboxylase inhibitor is carbidopa.

6. The method according to claim 1, wherein the motor dysfunction is caused by Parkinson's disease.

7. The method according to claim 1, wherein the motor dysfunction is tremor, bradykinesia, or rigidity.

8. The method according to claim 1, wherein the catechol-O-methyltransferase inhibitor, dopamine precursors and the peripheral decarboxylase inhibitor are administered in a fixed combination.

9. The method according to claim 1, wherein the mammal has not been previously treated with a dopamine precursor.

10. The method according to claim 1, wherein the mammal is human.
